(19) United States
(12) Patent Application Publication
(10) Pub. No.: US 2006/0122626 A1
(43) Pub. Date: Jun. 8, 2006

(54) Clamp for performing circumcisions on newborns and a method of using the same

(76) Inventor: Barry P. Duel, Long Beach, CA (US)

Correspondence Address: MYERS DAWES ANDRAS & SHERMAN, LLP, 19900 MACARTHUR BLVD.,, SUITE 1150, IRVINE, CA, 92612, US

(21) Appl. No.: 11/009,213

(22) Filed: Dec. 8, 2004

(51) Int. Cl. A61B 17/32

(52) U.S. Cl. 606/118

(57) ABSTRACT

A circumcision device is a unitary combination comprised of a body, a conical bell over which the foreskin is disposed and into which the glans is inserted, a clamping ring, and a shaft coupled to the body and to one the bell is connected. The device is made from an injection-molded plastic so that there is no need for hinges due to the resiliency of plastic. The clamping ring is split and moves between an open and closed locked position. The bell is drawn up into the locked ring to clamp the foreskin. A handle is coupled to the device to position it. A handle is coupled to the shaft for moving the shaft relative to the body. A flange is connected to a collar on the shaft to drive the clamp down onto the bell with a push of the thumb. A plurality of skin clips is coupled to one of either the body or shaft and fixed relative to the bell for temporarily holding the foreskin in place against the bell.

### RELATED APPLICATIONS

The present application is related to U.S. patent application Ser. No. 10/422,123 filed on Apr. 24, 2003, which is incorporated herein by reference.

### BACKGROUND OF THE INVENTION

1. Field of the Invention

The invention relates to surgical devices and methods, and in particular to a device and method for performing circumcisions on newborn infants.

2. Description of the Prior Art

In 2000, the most recent year for which complete data are available, there were 4,000,000 births in the U.S. Assuming that half of them were boys, and a 60% newborn circumcision rate (a somewhat conservative estimate), there are about 1,200,000 newborns circumcised in this country annually. All of these are done with some sort of clamp device. In addition, the Muslim world practices routine circumcision as well, which represents about another several million boys born annually.

All newborn circumcisions are done using some sort of clamp device. These serve to clamp the foreskin prior to cutting it, to prevent bleeding. The most popular devices are the Plastibell, Gomco and Mogen clamps. The Gomco clamp is generally felt to be safest, because it includes a bell to protect the glans, or head of the penis, during the clamping and cutting of the foreskin. However, it is cumbersome, and actually requires assembly on the patient each time it is used. The Mogen simply clamps the foreskin above the glans, and the excess skin is then removed. While it is easier to use than the Gomco, it is not quite as safe, and carries the risk of actually cutting or clamping part of the glans during the procedure, since there is no bell. The Plastibell is essentially a plastic ring which is placed inside the foreskin. A suture is then tied around it, the skin is excised, and the ring falls off in about one week.

The construction of the Gomco clamp and the development of the bloodless technique by Yellen in 1935 and Brodie in 1939 have encouraged the practice of routine circumcision. These factors have made the operation safer, reduced the chances of infection, and practically eliminated hemorrhage as compared to traditional surgical methods. The Gomco clamp is made up of four relatively heavy parts, which are difficult to manipulate and position during the operation, by a skilled surgeon or Mohel (a nonphysician trained in newborn circumcision consistent with Jewish tradition). The parts are a plate, a stud (bell), an arm (yoke), and a nut (to tighten the clamp). The device is thus expensive to manufacture and requires careful sterilization practices between multiple usages. The bell is introduced into the preputial cavity (over the glans and under the foreskin) and the prepuce is drawn over it. The plate is then placed over the bell so that the prepuce is sandwiched between them. The arm is fitted into its proper place, and when the nut is screwed on tightly, it exerts a crushing force on the prepuce at the junction of the bell and plate. The clamp is left on for 5 minutes to achieve hemostasis, and the prepuce is excised.

Another prior art device is called the Mogen clamp. Bronstein, a Brooklyn Mohel, who invented the Mogen clamp, also invented the Nutech clamp. The Mogen Clamp is a variation of the Kantor clamp. Instead of crushing the tissue by the attached hemostat, the Mogen shield acts as the clamp. It has the shortcomings of the Kantor clamp, plus one uniquely its own: with the glans below completely out of sight, there is a chance that the tip of the glans might be caught in the clamp. However, this is the preferred device of the more modern Jewish Mohel, and is newly reported to cause less discomfort during infant circumcision, than does the Gomco clamp.

The Mogen device is used by stretching the preputial opening and breaking the preputial adhesions so that the foreskin is completely retractile using a blunt-edged probe. The prepuce is lifted in an upward and outward direction with a hemostat. This action should cause the glans to retract towards the scrotum, preventing accidental amputation of the glans. The open jaws of the Mogen clamp are placed around the prepuce (grooved side facing the glans) and lifted upwards. The clamp is closed. It should be left closed for one to one and one-half minutes. If the infant is more than 6 months old it should remain closed for no less than five minutes. While the clamp is closed, the prepuce is excised distal to the clamp.

Yet another prior art device is the Plastibell made by Hollister. The Plastibell is a plastic bell with a groove close to the edge. It is a disposable plastic device. The bell is inserted into the preputial cavity (over the glans, and under the foreskin) and the foreskin (prepuce) is tied around it with a tight string. Blood flow to the prepuce is ceased, and the prepuce forward of the string is cut off. After several days, the prepuce caught under the string necrotizes and falls off, providing a bloodless circumcision, with no open wound to become irritated or infected.

While all circumcision devices are meant to simplify the operation and to increase the safety of the operation, use of the devices must nonetheless be done with knowledge of the limitations of the devices, which includes their appropriate use, particularly when being used by a single operator. For example, because of the danger of the plastic ring being pulled back behind the glans, only the foreskin that naturally covers the glans can be safely removed using the Plastibell. The Plastibell is primarily used on infants in the United States, although youth, teen and adult models are sometimes used in Europe. The Plastibell is used in nearly 60% of all routine infant circumcisions in the US with 39% being done by the Gomco clamp in the United States. Both the Gomco Clamp and the Plastibell, if used in accordance with the maker's recommendations, crush the foreskin. The biggest difference is that the Plastibell works entirely by this method whereas the Gomco crushes only until the unwanted skin is cut away to perform the actual circumcision. In theory either should produce equally cosmetic results.

The procedure is completed and the Plastibell is in place and will remain on the boy's penis for approximately 7 days before falling off (usually in the bath). Because the Plastibell remains on the penis for 5-10 days whilst it does its job, and is pulled tight against the glans by the foreskin trying to retract back to its unstretched position, Hollister recommends that the foreskin should not be pulled too far forward before being trapped by the thread around the Plastibell's groove. This results in a necessarily looser circumcision than can be achieved with the Gomco clamp. Furthermore, the position of the scar line behind the glans is determined only by the distance from the back of the glans to the groove in the bell. For a given glans circumference (and hence Plastibell size) the longer the boy's glans the further back the resulting scar line will be and the looser the circumcision will be.

If the Plastibell is fixed too tightly then the front of it can dig into the glans and cause problems, including obstruction of the urethra, because it is there for a week or so. The step of clamping the foreskin to the handle of the bell seems to me to be one which doctors are compelled to take if working alone—otherwise there is no way of ensuring that the foreskin stays in place over the bell whilst tying the knots to perform the circumcision proper. If there is an assistant doctor (or nurse) present then they can hold the end of the foreskin in forceps whilst the surgeon ties the knot. This step of keeping the foreskin pulled forward is essential if the Plastibell is to remove an adequate amount of foreskin.

However, the instructions for the Plastibell explicitly state that the skin should not be pulled too tight before being tied off. This is because if pulled very tight there will be considerable tension backwards after the bell has been tied in place. The end of the bell will be pulled into the glans and may easily compress the urethra to the extent of making urination very painful, if not impossible. A wise surgeon passes a probe down the urethra after fixing the bell so as to prove that it is not obstructed. Only then does he finally cut the thread to length.

As mentioned above, there are many different types of circumcision clamps. The Gomco is safest, since it incorporates a bell to protect the head of the penis (glans). However, it is very cumbersome, requiring assembly each time it is used. The Mogen is rapid and effective, but does not incorporate any method to protect the glans, and injuries are possible with this device, some quite severe. The Plastibell is safe and effective, but requires that a large stitch and plastic ring remain on the child's penis for 7-10 days. Understandably, many parents find this distressing.

What is needed is some is some type of clamp that has the advantages of the prior art devices describe above, but which is pre-assembled, making it much easier to use than, and which is safer than a Mogen clamp. In addition, it should include an integrated cutting blade, eliminating the additional need for a scalpel.

### BRIEF SUMMARY OF THE INVENTION

The invention is an apparatus for performing circumcision of a foreskin. The apparatus comprises a body, a conical bell over which the foreskin is disposed and into which the glans is inserted, a clamping ring, and a body coupled to the shaft and the shaft coupled to one of either the bell or clamping ring. Regardless of whether the bell is fixed to the body or the shaft, the variable disposition between the bell and the clamping ring provides for varying clearance between the clamping ring and bell to allow for clamping of the foreskin against the bell to achieve hemostasis. In other words, the bell can be held fixed and the clamping ring brought by the shaft downward into contact with the outer conical surface of the bell to clamp the foreskin between them; or the clamping ring can be held fixed and the bell drawn upwardly by the shaft into contact with the clamping ring to clamp the foreskin between the ring and bell. In the preferred embodiment the clamping ring is stationary and bell is moved by means of the shaft. Where the clamping ring is stationary and the bell moves, the clamping ring is split and moves between an open and closed locked position. However, where the bell is stationary and the clamping ring moves, it is possible that the clamping ring will be a fixed or closed circle.

The body, bell, clamping ring and shaft comprise a unitary combination during use, i.e. it normally does not need to be assembled or disassembled to be used.

Preferably, the bell is coupled to the shaft and a handle is fixed to the body to facilitate positioning of the body with a single hand. A handle is coupled to the shaft for moving the shaft relative to the body, and a flange is coupled to the collar for moving the collar relative to the shaft, allowing the clamp to be driven down onto the bell using only one hand. In the preferred embodiment, a hand held scalpel is used, but it is to be understood that a knife assembly could be rotatably coupled to the shaft to cut the foreskin.

A plurality of skin clips could be coupled to one of either the body or shaft and fixed relative to the bell for temporarily holding the foreskin in place against the bell.

In the illustrated embodiment the clamping ring is comprised of two semicircular clamps that include a catch for locking the two semicircular clamps together.

The body, bell, clamping ring and shaft combine to form a disposable or nondisposable combination, made preferably out of an injection molded plastic, such as polycarbonate. Due to the flexing capabilities of plastic, the clamping ring arms do not require hinges to be coupled to the collar. The injection-molded plastic allows the clamping arms to flex downward, allowing the two semicircular clamps to be pressed together and lock forming the clamping ring.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side elevational view of the illustrated embodiment of the invention shown with the elements of the device in an open or unengaged configuration with respect to the foreskin to be removed.

FIG. 2 is a side elevational view of the illustrated embodiment of the invention in FIG. 1 shown in the closed position or the engaged configuration with respect to the foreskin to be removed.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

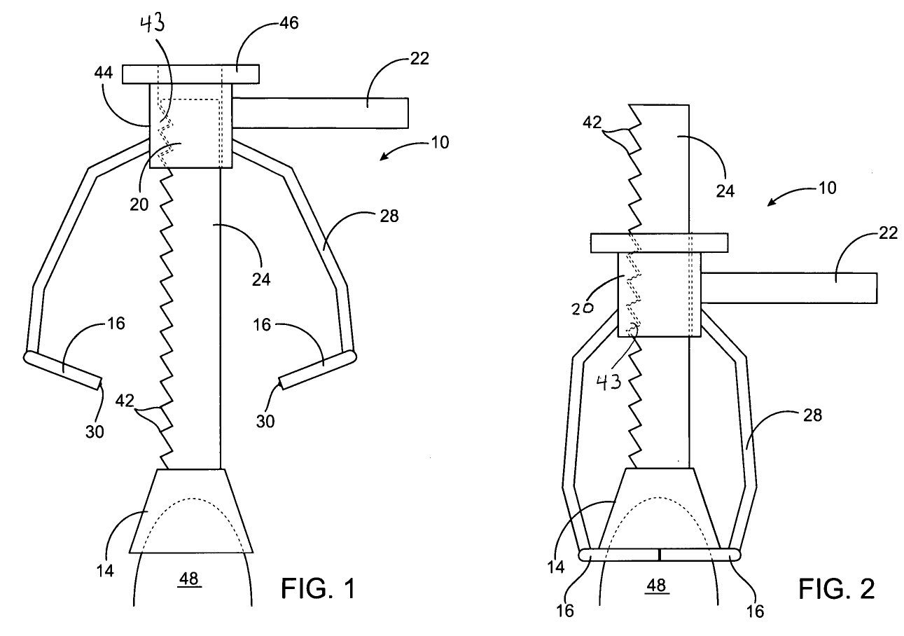

The circumcision clamp of the invention, known as the Duel Clamp named after its inventor, and generally denoted by reference numeral 10, is shown in side elevational view in FIG. 1 in an open or unengaged configuration. Clamp 10 is comprised of a body 20 from which a fixed handle 22 extends. A body 20 moves in relation to a shaft 24 that is engaged in a bore defined in body 20. The body 20 can be moved with respect to shaft 24 to vertically advance or retract relative to the shaft 24. In the preferred embodiment, shaft 24 is ratcheted for this purpose, while any other mechanism or arrangement may be equivalently substituted such as a rack and pinion combination, slide-and-wedge combination or any other means for relative movement now known or later devised. The ratcheting assembly that couples the collar 44 to shaft 24 is conventional and includes any type of one-way releasable mechanism, such as a spring biased set of ratcheting teeth 42 defined on shaft 24 and mating teeth 43 defined in collar 44, which teeth 42 and 43 are urged together by means of a sliding leaf spring (not shown) carried on collar 44 opposing the side of shaft 24 on which teeth 42 are defined, which spring urges teeth 42 and 43 into engagement, but when radially depressed allows teeth 42 and 43 to become disengaged. Alternatively, the resiliency of the plastic material of which collar 44 and shaft 24 are made may be sufficient for a simple two-piece ratchet structure to be provided, namely collar 44 may be easily moved downward by deforming teeth 42 and 43 over each other, but the angle and shape of teeth 42 and 43 is such that upward movement is not reasonable possible except in the case of excessive force, much like the ratcheting action of a conventional plastic tie. The ratcheting assembly need not be releasable since the device 10 may be disposable and can be removed with the foreskin held in ring 16 after being cut and then simply discarded. Further, for the purposes of this specification and claims, the term “ratcheting assembly” shall be defined to include any mechanism for allowing one way movement, and shall not be strictly restricted to a ratchet mechanism.

As shown in FIG. 1, shaft 24 is coupled to an elongated handle 22 which works in combination with a protruding flange 46 coupled to the collar 44, allowing for the handle 22 of the shaft 24 to be held with the user's fingers, and the semicircular clamps 16 to be driven down onto a bell 14 with a push of the user's thumb of the same hand. Handle 22 may have any form desired to facilitate the positioning or holding of clamp 10 during use. In the illustrated embodiment they include a horizontal fixed bar to assist in firm gripping or engagement of handle 22 with the fingers of one hand. A pair of clamp arms 28 are coupled to body 20 at their upper end as shown in FIG. 1 and carry a corresponding pair of fixed semicircular clamps 16 at their opposing lower end. One or both of clamp arms 28 are adjustable about their upper coupling to body 20. The lower ends of clamp arms 28 are each fixed to one of the semicircular clamps 16. The opposing end of the semicircular clamps 16 are provided with a catch 30, so that when semicircular clamps 16 are moved, flexed or pressed together, catch 30 engages on each opposing end of the semicircular clamps 16 and locks semicircular clamps 16 into the configuration of a rigid closed circle or ring.

In an alternate embodiment, clamp arms 28 may be spring-biased toward their open position by conventional means, may be freely adjustable relative to body 20 but friction stabilized, or may be resiliently coupled to a sliding collar 44.

The one-way releasable mechanism allows the collar 44, clamp arms 28 and semicircular clamps 16 to move down the shaft 24, but remain in the lowermost position obtained. In the preferred embodiment, the clamp 10 is made from a clear injection-molded plastic material, preferably polycarbonate. Due to the flexible characteristics of plastic material, the arms 28 are formed within the same mold as the collar 44 and do not need the assistance of hinges to close and form a clamping ring. The arms 28 are moved together by an outside manual force provided by the user, causing the clamps 16 to form a semicircle and the catch 30 locks the semicircle together.

A hollow bell 14 is coupled to the lower end of shaft 24 as seen in FIG. 1 to fit over the head of a penis 48. Semicircular clamps 16 are positioned relative to the throw of collar 44 on shaft 24 such that bell 14 is disposed inside of the rigid closed circle or ring of semicircular clamps 16 at variously adjustable vertical positions as best seen FIG. 2. Bell 14 has a truncated conical shape so that the amount of clearance between bell 14 and the rigid closed circle or ring of semicircular clamps 16 can be adjusted by adjusting the throw of shaft 24 and hence the vertical position of bell 14 in the rigid closed circle or ring of semicircular clamps 16.

In the preferred embodiment, a hand held scalpel is used to bear against the foreskin stretched over the bell 14 and held in place by means of the rigid closed circled or ring of semicircular clamps 16 as described below. Alternatively, a pair of knife assemblies each having its own knife edge could be employed and coupled to the shaft 24 to each cut at least an 180° incision in the case where a 360° were not possible due to the interference of clamp arms 28. It is of course to be understood that a single knife assembly could be used where a 360° incision is possible.

The most difficult part of assembling a Gomco clamp is pulling the bell and foreskin through the hole in the baseplate, then assembling the entire device. The clamp 10 of the invention eliminates these problems. First, the clamp 10 is pre-assembled, making its use much easier. Second, the baseplate of the Gomco is replaced with the semicircular clamps 16, which then clamp around the foreskin and tighten down to the bell 14. This makes for a much quicker procedure.

Clamp 10 may be a disposable plastic unit or a non-disposable sterilizable metallic unit. In the preferred embodiment, the clamp is made from an injection-molded plastic, such as polycarbonate. In either case, clamp 10 is integral in the sense that no element or piece has to be assembled in order for clamp 10 to be used or the operation using clamp 10 performed. Clamp 10 is held in place with one hand during the operation and clamping action of semicircular clamps 16 and bell 14, and a hand-held scalpel is used in the other hand. In the case of a non-disposable version, the semicircular clamps 16 and bell 14 could be removable and manufactured in different sizes, which would reduce costs versus an entirely disposable version. The advantage of the injection-molded plastic unit is that due to the flexibility characteristics of plastic, the clamp 10 does not require any hinges to attach the clamp arms 28 to the collar 44. The clamps 16 are pressed around bell 14 by an outside manual force provided by the user.

The structure of clamp 10 having been described above, consider now the use of clamp 10. For use, a slit is made in the dorsal surface of the foreskin, and bell 14 is placed over the glans within the foreskin. The semicircular clamps 16 are then brought together locking in place with catch 30, forming a complete circle around the foreskin, analogous to the baseplate of a Gomco clamp. Hemostasis is then achieved in this locked configuration. In the preferred embodiment, clamp arms 28 are directly attached to a sliding collar 44 on the shaft 24. An outside manual force, for example by the user's free hand forces the semicircular clamps 16 and bell 14 together, pinching the foreskin circumferentially. Arms 28 are moved downward and the halves of ring 16 are brought together to form a circle and to lock catch 30.

In an alternative embodiment, a conventional cam mechanism such as a cam (not shown) is connected to handle 22 which bears against follower cam surfaces (not shown) provided on the upper ends of arms 28 to cause arms 28 to move downward as handle 22 is pushed downward.

Once the arms 28 are locked by catch 30, and the bell is on the foreskin, a hand-held scalpel is then brought to bear the foreskin for the incision. The semicircular clamps 16 are then released, and the entire clamp 10 is removed. Thus clamp 10 is used as a unitary device during the operation and is disengaged from the patient as a unitary device. In the case of the Gomco device, the unit must be held by an assistant at some points during the operation or left attached unsupported hanging from the patient. Given the size and weight of the Gomco device and the small size of newborns, this option is often impractical or undesirable.

The preferred embodiment has been shown in and described in FIGS. 1 and 2, including a bell 14 and an openable and closeable clamping ring 16 whose position is controlled by the movement of the surgeon's hand gripping handle 22, to move ring 16 downwardly to cinch up against bell 14. The relative roles of bell 14 and clamping ring 16 could also be reversed without departing from the spirit and scope of the invention. For example, the surgeon may hold shaft 24 by means of handle 22 and position bell 14 in place with one hand and move clamping ring 16 with the other hand to cinch up against bell 14. Ring 16 in this embodiment may be a fixed or unbroken ring which is cinched up against bell 14 by being movably connected to shaft 24 by arms 28 with which it may be integral. The longitudinal position of ring 16 relative to the handle 22 is adjusted by means of a ratcheting, threaded, or sliding connection between arms 28 and shaft 24. Arms 28 are fixed to shaft 24 and there is no need for a swivel joint between the bell 14 and the shaft 24. In the embodiment of FIGS. 1 and 2 ring 16 is brought into operative engagement with the foreskin on bell 14 by means of one hand which also serves to hold device 10 as a whole in place. This leaves the other hand of the surgeon free to hold or stretch the foreskin in position over bell 14 as ring 16 is being brought to its closed position or to handle the scalpel or other cutting instrument.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for performing circumcision of a foreskin and adapted for unassisted use by a user comprising:
a body including a one-way releasable mechanism;
a conical bell over which the foreskin is disposed;
a clamping ring; and
a shaft coupled to the body and to one of either the bell or clamping ring for providing variable relative disposition between the bell and the clamping ring, the other one of the bell or clamping ring being coupled to the body by the one-way releasable mechanism, the clamping ring and bell being variably disposable relative to each other to provide for varying clearance between the clamping ring and bell to allow for clamping of the foreskin against the bell to achieve hemostasis, and where the body, bell, clamping ring and shaft comprise a unitary combination during use.

2. The apparatus of claim 1, wherein the bell is coupled to the shaft, and the clamping ring is coupled to the body.

3. The apparatus of claim 1, wherein the one-way releasable mechanism is one of a ratchet, threaded or sliding mechanism.

4. The apparatus of claim 1, wherein the body is directly coupled to the shaft, and the bell is movable.

5. The apparatus of claim 1, wherein the bell is coupled to the shaft, and the body is moveable.

6. The apparatus of claim 1 further comprising a handle coupled to the body to facilitate positioning of the bell with a single hand.

7. The apparatus of claim 1, further comprising a handle coupled to the shaft for moving the body relative to the shaft

8. The apparatus of claim 2, where the clamping ring is comprised of two semicircular clamps.

9. The apparatus of claim 1, where the clamping ring is comprised of two movable clamp arms, each coupled to one of the two semicircular clamps.

10. The apparatus of claim 9, where the two semicircular clamps include a catch for locking the two semicircular clamps together.

11. The apparatus of claim 1, where the body, bell, clamping ring and shaft combine to form a disposable combination.

12. The apparatus of claim 1, where the body, bell, clamping ring and shaft combine to form a nondisposable combination.

13. The apparatus of claim 1, further comprising a collar coupled to the shaft for moving the clamping arms relative to the body.

14. The apparatus of claim 1, further comprising a flange coupled to the collar to facilitate positioning of the body with a single hand.

15. The apparatus of claim 1, wherein the shaft operates on a sliding ratchet assembly.

16. The apparatus of claim 1, wherein the sliding ratchet assembly allows for direct coupling between the bell and the shaft.

17. The apparatus of claim 1, wherein the apparatus is made from an injection molded plastic.

18. The apparatus of claim 17, wherein the apparatus is made from polycarbonate.

19. The apparatus of claim 1, wherein the clamp arms are directly coupled to the collar.

20. The apparatus of claim 1, wherein the clamping ring is comprised by resiliently flexing the movable clamp arms.

21. An apparatus for performing circumcision of a foreskin and adapted for unassisted use by a user comprising:
a body;
a handle coupled to the body to facilitate positioning of the body with a single hand;
a pair of clamp arms coupled to the body by a one-way releasable mechanism;
a pair of semicircular clamps, each one of the pair of semicircular clamps coupled to one of the pair of clamp arms;
a shaft coupled to the body;
a handle coupled to the body for moving the shaft relative to the body; and
a conical bell coupled to the shaft over which the foreskin is disposed, where the shaft provides for variable disposition between the bell and the pair of semicircular clamps for varying clearance between the clamping ring and bell to allow for clamping of the foreskin against the bell to achieve hemostasis, and where the body, bell, clamping ring and shaft comprise a unitary combination during use.

22. The apparatus of claim 21, where relative movement of the shaft with respect to the body opens or closes the pair of arms to open or close the pair of semicircular clamps.

23. The apparatus of claim 21, wherein the one-way releasable mechanism is one of a ratchet, threaded or sliding mechanism.

24. The apparatus of claim 21, where the two semicircular clamps includes a catch for locking the two semicircular clamps together.

25. The apparatus of claim 21, where the body, bell, clamping ring and shaft combine to form a disposable combination.

26. The apparatus of claim 21, where the body, bell, clamping ring and shaft combine to form a nondisposable combination.

27. The apparatus of claim 21, further comprising a collar coupled to the shaft for moving the clamping arms relative to the shaft.

28. The apparatus of claim 21, further comprising a flange coupled to the collar to facilitate positioning of the body with a single hand.

29. The apparatus of claim 21, wherein the shaft operates on a sliding ratchet assembly.

30. The apparatus of claim 21, wherein the sliding ratchet assembly allows for direct coupling between the bell and the shaft.

31. The apparatus of claim 21, wherein the apparatus is made from an injection molded plastic.

32. The apparatus of claim 21, wherein the apparatus is made from polycarbonate.

33. The apparatus of claim 21, wherein the clamp arms are directly coupled to the collar.

34. The apparatus of claim 21, wherein the clamping ring is closed and opened by resiliently flexing the movable clamp arms.
